(19) United States
(12) Patent Application Publication
(10) Pub. No.: US 2001/0041706 A1
(43) Pub. Date: Nov. 15, 2001

(54) Blockade of taxane metabolism

(76) Inventors: Timothy W. Synold, Monrovia, CA (US); James H. Doroshow, Arcadia, CA (US)

Correspondence Address: ROTHWELL, FIGG, ERNST & MANBECK, P.C., 555 13TH STREET, N.W., SUITE 701, EAST TOWER, WASHINGTON, DC, 20004, US

(21) Appl. No.: 09/814,072

(22) Filed: Mar. 22, 2001

Related U.S. Application Data
(60) Provisional application No. 60/191,828, filed on Mar. 24, 2000.

(51) Int. Cl. A61K 35/78; A61K 31/7048; A61K 31/496; A61K 31/337

(52) U.S. Cl. 514/254.07; 424/736; 514/27; 514/449; 514/456

(57) ABSTRACT

The present invention relates to methods of inhibiting taxane metabolism in patients receiving taxane treatment, in which an effective amount of a CYP3A4 inhibitor and a CYP2C8 inhibitor are administered to the patient.

### CROSS-REFERENCE TO RELATED APPLICATIONS

This application claims the benefit of application Ser. No. 60/191,828 filed Mar. 24, 2000.

### STATEMENT REGARDING FEDERALLY SPONSORED RESEARCH OR DEVELOPMENT

This invention was made in part under grant No. CA 62505 from the United States National Cancer Institute. The United States government has certain rights in the invention.

### BACKGROUND OF THE INVENTION

1. Field of the Invention

The present invention relates to methods of inhibiting taxane metabolism in a patient receiving taxane treatment, in which the patient is given an effective amount of a CYP3A4 inhibitor and a CYP2C8 inhibitor.

2. Description of the Related Art

Paclitaxel (sold under the TAXOL® brand by Bristol-Myers Squibb) is a taxane which exhibits wide patient-to-patient variability in elimination, particularly at higher doses. We have shown previously that the measured paclitaxel concentration in blood is a better predictor of drug-induced toxicity than the actual administered doses. Unfortunately, there is currently no way to accurately predict a priori which patients will have higher drug levels, and therefore, worse toxicity. To illustrate the importance of paclitaxel pharmacokinetic variability, we have demonstrated that by individualizing paclitaxel doses based on each patient's measured drug levels, we can significantly decrease the variation in both pharmacokinetics and toxicity. This idea of “adaptive control” of individual paclitaxel systemic exposures, while clearly effective, is labor intensive and not readily transportable to centers that do not have specialized pharmacologic laboratories. Therefore, we have explored another approach aimed at decreasing paclitaxel's pharmacokinetic variability by inhibiting the known routes of metabolism.

Paclitaxel is metabolized in the liver by two routes, CYP3A4 and CYP2C8. CYP3A4 represents approximately 25% of all cytochrome P450's in the human liver, and is the major route of oxidative metabolism for most drugs. While CYP2C8 is a relatively uncommon route of hepatic oxidative metabolism, it is the major pathway of paclitaxel inactivation. Although there are many known substrates for CYP3A4, very few specific substrates of CYP2C8 have been identified. Besides paclitaxel, only retinoic acid has been shown to be a substrate for CYP2C8. However, it has been previously reported that certain flavanoids found in foods, such as quercetin, kaempherol, and naringenin, can selectively inhibit the metabolism of paclitaxel by CYP2C8.

Fluorouracil, like paclitaxel, displays wide interpatient variability as a result of differences in dihydropyrimidine dehydrogenase (DPD) activity. Combination studies with DPD inhibitors and fluorouracil have shown that pharmacokinetics variability and doses required to achieve equivalent systemic exposure to single agent fluorouracil are significantly reduced. Whether clinically significant inhibition of paclitaxel metabolism is possible in humans was unclear prior to the present invention. Jamis-Dow and colleagues (Am J. Clin. Oncol. 1997) were unable to inhibit paclitaxel metabolism in humans using ketoconazole despite a documented 60% decrease in CYP3A4 activity. The authors suggested that the most likely explanation for why they were unable to inhibit paclitaxel elimination was that an inhibitor of CYP2C8 was not used because “. . . no clinically-usable inhibitors of CYP2C8 have been reported.” However, as previously mentioned, the ability of quercetin to selectively inhibit the CYP2C8 metabolism has been demonstrated, and quercetin is now commercially available as a dietary supplement.

### BRIEF SUMMARY OF THE INVENTION

In one aspect, the present invention relates to a method of inhibiting taxane metabolism in a patient receiving taxane treatment, which method comprises administering to said patient an effective amount of a CYP3A4 inhibitor and a CYP2C8 inhibitor.

In another aspect, the present invention relates to a pharmaceutical composition which comprises:

a) an effective amount of a CYP3A4 inhibitor;

b) an effective amount of a CYP2C8 inhibitor; and

c) a pharmaceutically acceptable carrier.

### BRIEF DESCRIPTION OF THE DRAWINGS

FIGS. 1 and 2 are bar graphs of results obtained in experiments to determine inhibition of paclitaxel metabolism by ketoconazole and quercetin in human liver micorosomes from two different donors.

FIGS. 3 and 4 are bar graphs of results obtained in experiments to determine inhibition of paclitaxel metabolism by ketoconazole and quercetin in primary human hepatocytes.

### DETAILED DESCRIPTION OF THE INVENTION

Taxanes are compounds which exhibit antitumor effect, and hence are used as chemotherapeutic agents. As mentioned above, the present invention relates to inhibition of taxane metabolism in patients receiving taxane treatment. Many taxanes are known and applicable to this invention, including paclitaxel, docetaxel, and other taxane analogs.

Inhibition of taxane metabolism may be achieved by administering an effective amount of a CYP3A4 inhibitor and a CYP2C8 inhibitor to a patient receiving taxane treatment. Suitable CYP3A4 inhibitors include ketoconazole, amiodarone, anastrozole, azithromycin, cannabinoids, cimetidine, clarithromycin, clotrimazole, cyclosporine, danazol, delavirdine, dexamethasone, diethyldithiocarbamate, diltiazem, dirithromycin, disulfiram, entacapone (high dose), erythromycin, ethinyl estradoil, fluconazole (weak), fluoxetine, fluvoxamine, gestodene, grapefruit juice, indinavir, isoniazid, itraconazole, metronidazole, mibefradil, miconazole (moderate), nefazodone, nelfinavir, nevirapine, norfloxacin, norfluoxetine, omeprazole (weak), oxiconazole, paroxetine (weak), propoxyphene, quinidine, quinine, quinupristin and dalfopristin, ranitidine, ritonavir, saquinavir, sertindole, sertraline, troglitazone, troleandomycin, valproic acid (weak), verapamil, zafirlukast and zileuton. Suitable CYP2C8 inhibitors include various flavanoids found in foods, such as quercetin, kaempherol, and naringenin; retinoic acid, carbamazepine, tolbutamide, sulfaphenazole, mephenytoin, etc., with quercetin being particularly preferred. The effective amount of the inhibitors to be administered will depend on a number of factors, such as particular inhibitors employed, amount of taxane given, rate of taxane administration (e.g., 1-hour, 3-hour, 24-hour infusion, etc.), route of taxane administration (e.g., i.v. or oral), etc. One of ordinary skill could readily determine an effective amount without undue experimentation. In general, for example, a dose of 400-800 mg of ketoconazole and 1-4 grams of quercetin will be effective in conjunction with paclitaxel therapy. The inhibitors are administered to the patient in conjunction with taxane therapy. The inhibitors may be administered prior to the taxane, for example up to about 24 hours before commencing taxane therapy. The inhibitors may be administered concurrently with the taxane, and may even be administered up to about 72 hours after completion of taxane administration, depending on how complete the inhibitation is. The inhibitors can be, but need not be administered to the patient at the same time. The inhibitors may be administered by any suitable route, for example orally, parenterally, intravenously, etc.

Also within the scope of the present invention are pharmaceutical compositions which contain an effective amount of a CYP3A4 inhibitor and/or a CYP2C8 inhibitor, as well as one or more pharmaceutically acceptable carriers. Such compositions include both solid (capsules, tablets, etc.) and liquid (solutions, suspensions, etc.) forms.

The use of the present invention may be further illustrated by reference to the following non-limiting examples.

### EXAMPLE 1

Armed with the knowledge that paclitaxel is metabolized by two major routes, and that there are specific inhibitors of each pathway, we performed a series of mouse pharmacokinetic studies designed to determine if paclitaxel elimination could be significantly decreased by co-administration of known inhibitors of CYP3A4 and CYP2C8 in vivo.

Paclitaxel pharmacokinetcis were studied in female FVB1 mice receiving 10 mg/kg by tail vein injection on the following schedules:

a) paclitaxel alone;

b) paclitaxel one hour after ketoconazole 100 mg/kg by gavage;

c) paclitaxel one hour after quercetin 1000 mg/kg by gavage;

d) paclitaxel one hour after ketoconazole 100 mg/kg and quercetin 1000 mg/kg by gavage;

e) paclitaxel twelve hours after quercetin 1000 mg/kg by gavage; and

f) paclitaxel 12 hours after quercetin 1000 mg/kg by gavage and one hour after ketoconazole 100 mg/kg by gavage.

At multiple time points after administration of paclitaxel, three mice were euthanitized, bled, and the livers were collected. Paclitaxel and quercetin in plasma and liver were determined by HPLC.

Pretreatment with ketoconazole alone resulted in 59% increase in paclitaxel area under the curve (AUC) (p, 0.05), whereas pretreatment with quercetin one hour prior to paclitaxel had no effect. The combination of ketoconazole and quercetin one hour before paclitaxel resulted in a 146% increase in pharmacokinetic system exposure (p<0.05). Because HPLC analysis of quercetin in liver indicated that quercetin levels were high starting 8-12 hours after an oral dose, mice were treated on schedules e and f above. Quercetin twelve hours prior to paclitaxel had no effect on system exposure. However, the combination of ketoconazole and quercetin increased paclitaxel AUC by 104% (p<0.05). The peak levels of quercetin measured in the livers of mice receiving oral supplementation were approximately 15 μM, and occurred between 8 and 12 hours after the dose.

These data demonstrate that combination pretreatment with oral ketoconazole and quercetin can significantly decrease paclitaxel clearance and increase AUC in vivo. This may allow the use of lower doses of paclitaxel to achieve similar system exposure, while decreasing interpatient pharmacokinetic variablity.

### EXAMPLE 2

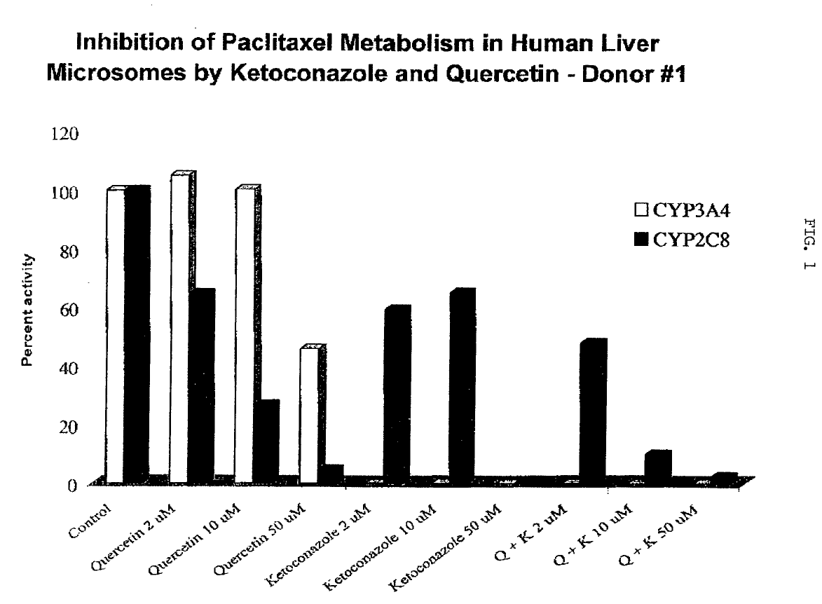

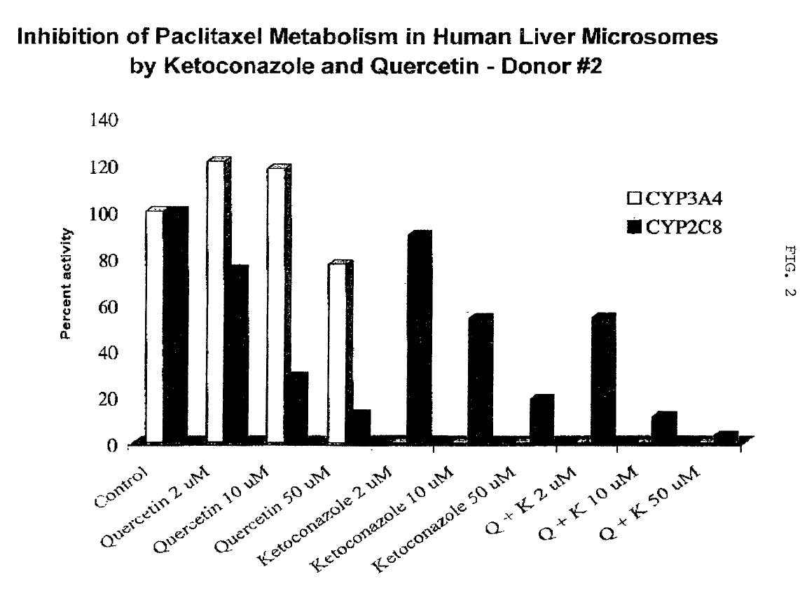

We subsequently performed experiments in human liver mocrosomes designed to confirm that ketoconazole and quercetin can inhibit paclitaxel metabolism by human CYP3A4 and CYP2C8, respectively. FIGS. 1 and 2 show results from two different human hepatocyte donors. In addition to confirming the selective effects of single agent ketoconazole and quercetin, the results of these studies demonstrate that ketoconazole and quercetin in combination at concentrations achievable in vivo completely inhibit CYP3A4 and inhibit CYP2C8 by 85%.

### EXAMPLE 3

Two identical experiments were performed in which primary human hepatocytes isolated from cadaver donors were incubated with paclitaxel and increasing concentrations of ketoconazole, quercetin, or the combination for 24 hours. At the end of the incubation, the media was removed and assayed for paclitaxel and paclitaxel metabolite concentrations. The formulation of 3OH-paclitaxel represents metabolism of paclitaxel by cytochrome P450 3A4 (CYP3A4) and the formation of 6OH-paclitaxel represents metabolism by cytochrome P450 2C8 (CYP2C8).

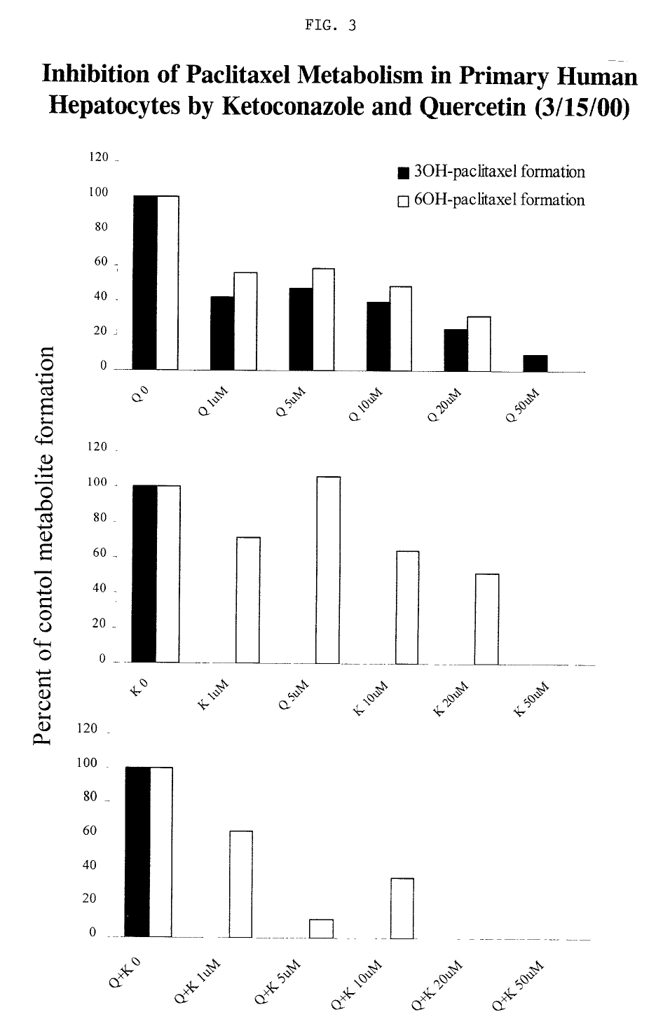

The results of the first experiment, shown in FIG. 3, demonstrate that 1) quercetin was able to inhibit paclitaxel metabolism of CYP3A4 and CYP2C8 in a concentration-dependent manner; 2) ketoconazole completely inhibited paclitaxel metabolism by CYP3A4 starting at concentrations of 1 μM and metabolism by CYP2C8 starting at 50 μM; and 3) the combination of ketoconazole and quercetin inhibited paclitaxel metabolism better than either drug alone, and completely inhibited metabolism at 20 μM.

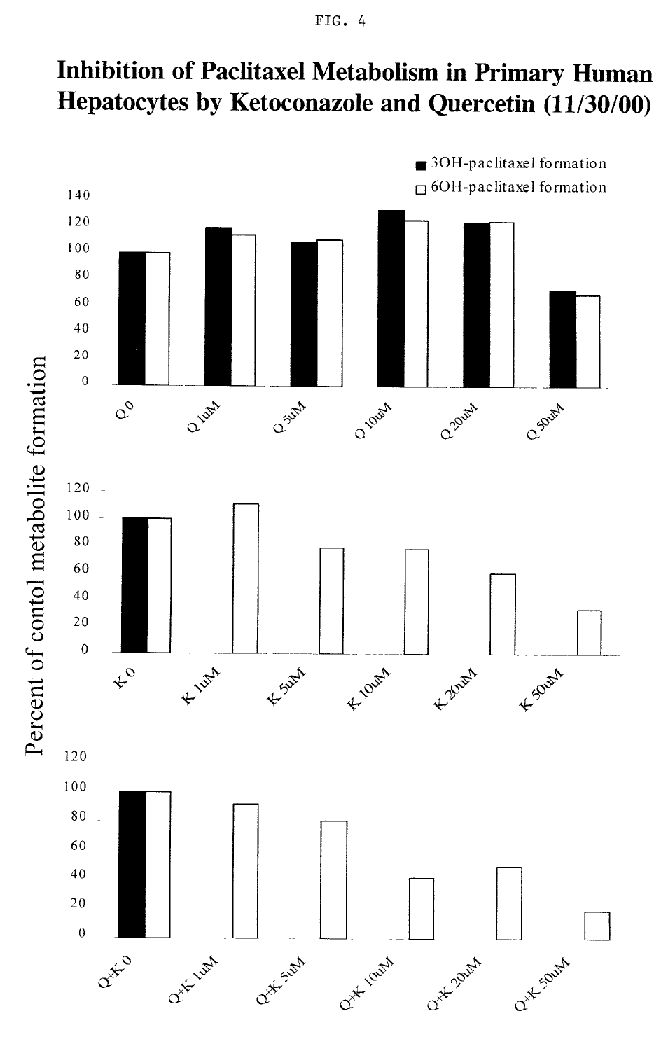

The results of the second experiment, shown in FIG. 4, demonstrate that 1) quercetin had a modest effect on paclitaxel metabolism at the highest concentration tested (50 μM); 2) ketoconazole completely inhibited paclitaxel metabolism by CYP3A4 starting at concentrations of 1 μM and inhibited metabolism by CYP2C8 in a concentration-dependent manner; and 3) the combination of ketoconazole and quercetin inhibited paclitaxel metabolism to a greater extent than ketoconazole alone.

In summary, the data generated to date demonstrate the feasibility of significantly inhibiting paclitaxel elimination in vivo. Currently, paclitaxel is administered clinically at fixed doses based upon a patient's body surface area, and without any concern for the wide patient-to-patient variation in systemic exposure. By inhibiting paclitaxel elimination to the same extent in all patients, it is anticipated that the patient-to-patient variability in pharmacokinetics will be greatly decreased, and thus make clinical dosing of this toxic agent more predictable. Moreover, by significantly inhibiting paclitaxel elimination, there will be an additional benefit of reducing the required dosage of a very expensive chemotherapeutic.

Paclitaxel is formulated in an ethoxylated castor oil available under the trade name Cremaphor EL, at a ratio of 1 mg of paclitaxel for every 87.8 mg of Cremaphor EL. Cremaphor EL has been associated with severe allergic reactions, such that all patients receiving paclitaxel require pre-medication with steroids and anti-histamines to prevent hypersensitivity reactions. Moreover, Cremaphor EL has been shown to be responsible, in part, for the non-linear and highly variable pharmacokinetics of paclitaxel in patients receiving standard doses of the drug. Pre-administration or co-administration of a CYP3A4 inhibitor may permit administration of lower doses of both paclitaxel and Cremaphor EL, and thus lower the incidence of severe allergic reactions and minimize the effects of the vehicle on paclitaxel pharmacokinetics.

1. A method of inhibiting taxane metabolism in a patient receiving taxane treatment, which method comprises administering to said patient an effective amount of a CYP3A4 inhibitor and a CYP2C8 inhibitor.

2. The method of claim 1, wherein the taxane comprises paclitaxel or docetaxel.

3. The method of claim 2, wherein the taxane comprises paclitaxel.

4. The method of claim 1, wherein the CYP3A4 inhibitor and the CYP2C8 inhibitor are administered prior to taxane treatment.

5. The method of claim 1, wherein the CYP3A4 inhibitor and the CYP2C8 inhibitor are administered concurrently with taxane treatment.

6. The method of claim 1, whereIn the CYP3A4 inhibitor and the CYP2C8 inhibitor are administered at the same time.

7. The method of claim 1, wherein the CYP3A4 inhibitor and the CYP2C8 inhibitor are administered at different times.

8. The method of claim 1, wherein the CYP3A4 inhibitor and the CYP2C8 inhibitor are administered subsequent to taxane treatment.

9. The method of claim 1, wherein the CYP3A4 inhibitor is selected from the group consisting of ketoconazole, amiodarone, anastrozole, azithromycin, cannabinoids, cimetidine, clarithromycin, clotrimazole, cyclosporine, danazol, delavirdine, dexamethasone, diethyldithiocarbamate, diltiazem, dirithromycin, disulfiram, entacapone, erythromycin, ethinyl estradoil, fluconazole, fluoxetine, fluvoxamine, gestodene, grapefruit juice, indinavir, isoniazid, itraconazole, metronidazole, mibefradil, miconazole, nefazodone, nelfinavir, nevirapine, norfloxacin, norfluoxetine, omeprazole, oxiconazole, paroxetine, propoxyphene, quinidine, quinine, quinupristin and dalfopristin, ranitidine, ritonavir, saquinavir, sertindole, sertraline, troglitazone, troleandomycin, valproic acid, verapamil, zafirlukast and zileuton, and combinations thereof.

10. The method of claim 9, wherein the CYP3A4 inhibitor comprises ketoconazole.

11. The method of claim 1, wherein the CYP2C8 inhibitor is selected from the group consisting of quercetin, kaempherol, naringenin, retinoic acid, carbamazepine, tolbutamide, sulfaphenazole, and mephenytoin, and combinations thereof.

12. The method of claim 11, wherein the CYP2C8 inhibitor comprises quercetin.

13. The method of claim 1, wherein the CYP3A4 inhibitor comprises ketoconazole and the CYP2C8 inhibitor comprises quercetin.

14. A pharmaceutical composition which comprises:
a) an effective amount of a CYP3A4 inhibitor;
b) an effective amount of a CYP2C8 inhibitor; and
c) a pharmaceutically acceptable carrier.

15. The composition of claim 14, wherein the CYP3A4 inhibitor is selected from the group consisting of ketoconazole, amiodarone, anastrozole, azithromycin, cannabinoids, cimetidine, clarithromycin, clotrimazole, cyclosporine, danazol, delavirdine, dexamethasone, diethyldithiocarbamate, diltiazem, dirithromycin, disulfiram, entacapone, erythromycin, ethinyl estradoil, fluconazole, fluoxetine, fluvoxamine, gestodene, grapefruit juice, indinavir, isoniazid, itraconazole, metronidazole, mibefradil, miconazole, nefazodone, nelfinavir, nevirapine, norfloxacin, norfluoxetine, omeprazole, oxiconazole, paroxetine, propoxyphene, quinidine, quinine, quinupristin and dalfopristin, ranitidine, ritonavir, saquinavir, sertindole, sertraline, troglitazone, troleandomycin, valproic acid, verapamil, zafirlukast and zileuton, and combinations thereof.

16. The composition of claim 15, wherein the CYP3A4 inhibitor comprises ketoconazole.

17. The composition of claim 14, wherein the CYP2C8 inhibitor is selected from the group consisting of quercetin, kaempherol, naringenin, retinoic acid, carbamazepine, tolbutamide, sulfaphenazole, and mephenytoin, and combinations thereof.

18. The composition of claim 17, wherein the CYP2C8 inhibitor comprises quercetin.

19. The composition of claim 14, wherein the CYP3A4 inhibitor comprises ketoconazole and the CYP2C8 inhibitor comprises quercetin.
